(19) United States
(12) Patent Application Publication
(10) Pub. No.: US 2025/0000842 A1
(43) Pub. Date: Jan. 2, 2025

(54) Formulation of Resiniferatoxin

(71) Applicant: Sorrento Therapeutics, Inc., San Diego, CA (US)

(72) Inventors: Bryan Jones, San Diego, CA (US); Alexis G. Nahama, San Diego, CA (US)

(73) Assignee: Sorrento Therapeutics, Inc., San Diego, CA (US)

(21) Appl. No.: 18/622,139

(22) Filed: Mar. 29, 2024

Related U.S. Application Data
(63) Continuation of application No. 17/569,340, filed on Jan. 5, 2022, now abandoned.
Application No. 17/569,340 is a continuation of application No. 16/128,053, filed on Sep. 11, 2018, now abandoned.
(60) Provisional application No. 62/556,824, filed on Sep. 11, 2017.

(51) Int. Cl. A61K 31/357; A61K 9/00; A61K 9/08; A61K 47/02; A61K 47/10; A61K 47/26; A61K 47/40

(52) U.S. Cl. CPC A61K 31/357; A61K 9/0019; A61K 9/08; A61K 47/02; A61K 47/10; A61K 47/26; A61K 47/40

(57) ABSTRACT

Disclosed herein are safer formulations of resiniferatoxin (RTX) for intrathecal, intraganglionic intraarticular and pericardial administration. More specifically, there is disclosed alcohol-free formulations of RTX comprising a solubilizing component, a monosaccharide or sugar alcohol, a saline buffer, and RTX, and having narrow ranges for pH range and specific gravity.

### CROSS REFERENCE TO RELATED APPLICATION

This application is a continuation of U.S. application Ser. No. 17/569,340, filed on Jan. 5, 2022, which is a continuation of U.S. application Ser. No. 16/128,053, filed on Sep. 11, 2018, which claims the benefit of priority of U.S. Provisional Application No. 62/556,824, filed on Sep. 11, 2017, the entire contents of each of which are incorporated by reference in its entirety herein.

### FIELD OF THE INVENTION

The present disclosure provides lower toxicity formulations of resiniferatoxin (RTX) for administration. As RTX is an extremely aqueous insoluble compound, the disclosed formulations provide a high concentration of RTX active ingredient in a formulation wherein very little liquid can be injected, such as intrathecal, intraganglionic, periganglionic, pericardial or within a joint cavity (intraarticular). More specifically, the present disclosure provides alcohol-free formulations of RTX comprising a solubilizing component, a monosaccharide or sugar alcohol, a saline buffer, and RTX.

### BACKGROUND

The transient receptor potential cation channel subfamily V member 1 (TrpV1) or (Vanilloid receptor-1 (VR1)) is a multimeric cation channel prominently expressed in nociceptive primary afferent neurons (Caterina et al. (1997) Nature 389:816-824; Tominaga et al. (1998) Neuron 531-543. Activation of TrpV1 typically occurs at the nerve endings via application of painful heat and is up regulated during certain types of inflammatory stimuli. Activation of TrpV1 in peripheral tissues by a chemical agonist results in the opening of calcium channels and the transduction of a pain sensation (Szallasi et al. (1999) Mol. Pharmacol. 56:581-587. However, direct application of certain TrpV1 agonists to the cell body of a neuron (ganglion) expressing TrpV1 opens calcium channels and triggers a cascade of events leading to programmed cell death (“apoptosis”) (Karai et al. (2004) Journal of Clinical Investigation. 113:1344-1352).

RTX is known as a TrpV1 agonist and acts as an ultrapotent analog of capsaicin, the pungent principal ingredient of the red pepper. RTX is a tricyclic diterpene isolated from certain species of Eurphorbia. A homovanillyl group is an important structural feature of capsaicin and is the most prominent feature distinguishing resiniferatoxin from typical phorbol-related compounds. Naturally occurring or native RTX has the following structure:

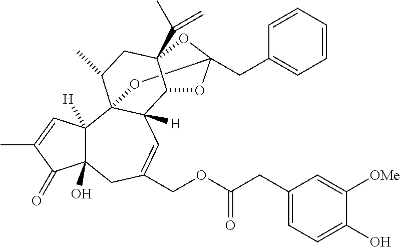

RTX and analog compounds such as tinyatoxin and other compounds, (20-homovanillyl sters of diterpenes such as 12-deoxyphorbol 13-phenylacetate 20-homovanillate and mezerein 20-homovanillate) are described in U.S. Pat. Nos. 4,939,194; 5,021,450; and 5,232,684. Other resiniferatoxin-type phorboid vanilloids have also been identified (Szallasi et al. (1999) Brit. J. Phrmacol. 128:428-434).

In U.S. Pat. No. 8,338,457 (the disclosure of which is incorporated by reference herein) RTX was diluted with 0.9% saline from a stock formulation, which contained 1 mg/mL of RTX, 10% ethanol, 10% Tween 80 and 80% normal saline. The vehicle that was injected was a 1:10 dilution of the RTX stock formulation using 0.9% saline as the diluent. Therefore, prior injections have dissolved the hydrophobic RTX molecule in ethanol and injected the formulation with about 1-2% (v/v) ethanol directly into the ganglion. However, it is inadvisable to inject ethanol (or other organic solvents) directly into the brain, spinal cord (subdural) or ganglion because these compounds can non-specifically kill any cell they come into contact with and nerves are particularly sensitive. Accordingly, there is a need in the art to develop a formulation of RTX for administration that does not contain any organic solvents (such as ethanol) and still will keep the RTX molecule in solution. The present disclosure was made to achieve such a non-alcohol formulation.

SUMMARY

The present disclosure provides a non-alcoholic formulation of RTX for injectable administration to a relatively small volume comprising from about 10 μg/mL to about 200 μg/mL RTX in a formulation having enough monosaccharide or sugar alcohol to keep the specific gravity between 1.0 and 1.3. RTX can be solubilized in at least one, or a mixture, of PEG (0-40%), polysorbate (0-5%) and cyclodextrin (0-5%) in an aqueous buffer solution with saline and a pH from about 6.5 to about 7.5 and contains an antioxidant.

Preferably, the formulation comprises from about 25-50 μg/mL RTX. Preferably, the monosaccharide or sugar alcohol is selected from the group consisting of dextrose, mannitol, and combinations thereof. Preferably, the solubilizing agent is selected from the group consisting of polysorbate (20, 60 or 80), polyethylene glycol (PEG100, 200 300 400 or 600), cyclodextrin, and combinations thereof. Preferably, the buffer is selected from the group consisting of phosphate buffer, acetate buffer, citrate buffer, and combinations thereof. Preferably, the formulation further comprises an antioxidant. More preferably, the antioxidant is selected from the group consisting of ascorbic acid, citric acid, potassium bisulfate, sodium bisulfate acetone sodium bisulfate, monothioglycerol, potassium metabisulfite, sodium metabisulfite, and combinations thereof.

### DETAILED DESCRIPTION

### Definitions

“Intraganglionic administration” is administration to within a ganglion. Intraganglionic administration can be achieved by direct injection into the ganglion and also includes selective nerve root injections, or periganglionic administration, in which the compound passes up the connective tissue sleeve around the nerve and enters the ganglion from the nerve root just outside the vertebral column. Often, intraganglionic administration is used in conjunction with an imaging technique, e.g., employing MRI or x-ray contrast dyes or agents, to visualize the targeted ganglion and area of administration. Administration volumes range from around 50 μl for administration directly into the ganglion to 2 ml for periganglionic administration around the ganglion.

The term “subarachnoid space” or cerebral spinal fluid (CSF) space incorporates the common usage refers to the anatomic space between the pia mater and the arachnoid membrane containing CSF.

“Intrathecal administration” is the administration of compositions directly into the spinal subarachnoid space. The volume for intrathecal administration in a human adult id from 2 to 50 μg.

“Intraarticular administration” is the injection of compounds in an aqueous solution into a joint cavity, such as the knee or elbow. The volume for intraarticular administration for a human adult knee is from 3 to 10 ml of volume and 5 to 50 μg of RTX. Knees of pediatric humans or veterinary (dog or cats) are lower and proportionate in volume to the relative sizes of each species knees.

The present disclosure provides a non-alcoholic formulation of RTX for intrathecal, intraarticular, intraganglionic or periganglionic administration comprising from about 10 μg/mL to about 200 μg/mL RTX in a formulation having enough monosaccharide to keep the specific gravity between 1.0 and 1.3. RTX can be solubilized in at least one, or a mixture, of PEG (0-40%), polysorbate (0-5%) and cyclodextrin (0-5%) in an aqueous buffer solution with saline and a pH from about 6.5 to about 7.5 and containing an antioxidant.

RTX may be injected directly into a ganglion or at the nerve root (intrathecal or intraganglionic) using standard neurosurgical techniques to create a temporary environment in a dorsal root or autonomic ganglion. RTX may also be injected directly into the intraarticular space to treat arthritis pain in that particular joint. Duration of the effect of the RTX may be longer than the period over which the temporary environment is maintained. Any dosage can be used as required and tolerated by the patient. Administration may be performed with the assistance of image analysis using MRI or x-ray contrast dyes, to provide for direct delivery to the perikarya. For example, the procedure can be performed in conjunction with procedures such as CAT scan, fluoroscopy, or open MRI.

For intraganglionic administration, a typical volume injected is from 50 to 300 microliters delivering a total amount of RTX that ranges from about 50 nanograms to about 50 micrograms. For intraarticular administration, a typical volume injected into an adult knee is from 3 ml to 10 ml, delivering a total amount of RTX from 5 ng to 50 μg. Often the amount administered is from 200 ng to 10 μg. RTX can be administered as a bolus or infused over a period of time, typically from 1 to 10 minutes.

For intrathecal administration, an amount from about 0.5 to 5 cc, often 3 cc are injected into the subarachnoid space. The total amount of RTX in the injected volume is usually from about 500 nanograms to about 200 micrograms. Often the amount administered is from 20 μg to 50 μg. RTX can be administered as a bolus or infused over a period of time, typically from 1 to 10 minutes.

TABLE 1
RTX Solution Formulations

| Formulation | | Component |
|---|---|---|
| Number | Formulation Components | Concentration |
| 1 | RTX | 200 μg/mL |
| | Polysorbate 80 | 7.0% w/v |
| | Dextrose | 0.8% w/v |
| | 30 mM Phosphate Buffer w/ 0.44% NaCl | 30 mM, pH 7.2 |
| 2 | RTX | 200 μg/mL |
| | Polyethylene Glycol 300 | 3.0% v/v |
| | Polysorbate 80 | 0.1% w/v |
| | Dextrose | 0.8% w/v |
| | 10 mM Phosphate Buffer w/ 0.73% NaCl | 10 mM, pH 6.5 |
| 3 | RTX | 200 μg/mL |
| | Polyethylene Glycol 300 | 30.0% v/v |
| | Polysorbate 80 | 1.0% w/v |
| | 10 mM Phosphate Buffer w/ 0.86% NaCl | 10 mM, pH 6.5 |
| 4 | RTX | 200 μg/mL |
| | Polyethylene Glycol 300 | 30.0% v/v |
| | Polysorbate 80 | 0.04% w/v |
| | 10 mM Phosphate Buffer w/ 0.88% NaCl | 10 mM, pH 6.5 |
| 5 | RTX | 200 μg/mL |
| | Polysorbate 80 | 3.0% w/v |
| | Dextrose | 0.8% w/v |
| | 30 mM Phosphate Buffer w/ 0.54% NaCl | 30 mM, pH 7.2 |
| 6 | RTX | 200 μg/mL |
| | Polysorbate 80 | 3.0% w/v |
| | Mannitol | 0.8% w/v |
| | 30 mM Phosphate Buffer w/ 0.54% NaCl | 30 mM, pH 7.2 |
| 7 | RTX | 200 μg/mL |
| | Polysorbate 80 | 7.0% w/v |
| | Mannitol | 0.8% w/v |
| | 30 mM Phosphate Buffer w/ 0.45% NaCl | 30 mM, pH 7.2 |
| 8 | RTX | 200 μg/mL |
| | Polyethylene Glycol 300 | 3.0% v/v |
| | Polysorbate 80 | 0.1% w/v |
| | Mannitol | 0.8% w/v |
| | 10 mM Phosphate Buffer w/ 0.74% NaCl | 10 mM, pH 6.5 |
| 9 | RTX | 200 μg/mL |
| | Polyethylene Glycol 300 | 3.0% v/v |
| | Polysorbate 80 | 0.1% w/v |
| | Dextrose | 3.0% w/v |
| | 10 mM Phosphate Buffer w/ 0.34% NaCl | 10 mM, pH 6.5 |
| 10 | RTX | 200 μg/mL |
| | Polyethylene Glycol 300 | 3.0% v/v |
| | Polysorbate 80 | 0.1% w/v |
| | Mannitol | 3.0% w/v |
| | 10 mM Phosphate Buffer w/ 0.36% NaCl | 10 mM, pH 6.5 |
| 11 | RTX | 200 μg/mL |
| | Polysorbate 80 | 0.03% w/v |
| | Dextrose | 0.05% w/v |
| | 30 mM Phosphate Buffer w/ 0.54% NaCl | 30 mM, pH 7.2 |

### EXAMPLE 1: PREPARATION OF FORMULATIONS

The formulations in Table 1 were prepared as follows, using as examples formulations 3 and 5. Formulation 3 was made by preparing a 30 mM, pH 7.2 phosphate buffer. Then 1.43% w/v polysorbate 80 and 0.86% w/v NaCl were mixed to form the aqueous component. 20 mg of RTX was added to 100 mL of the aqueous component in a volumetric flask. Then 30 mL of PEG 300 was added and the solution was sonicated to dissolve the solids. The aqueous component was added to about 80% volume, and then it was sonicated to mix. It should be noted that RTX will sometimes precipitate at the interface of aqueous solution and PEG initially, but will go back into solution upon sonication. The full mixture in the flask was diluted to volume with the aqueous component and this was mixed by an inversion process. The full formulation was filtered through a 0.2 μm polytetrafluoroethylene (PTFE) filter.

Formulation 5 was made by preparing 30 mM, pH 7.2 phosphate buffer. Then 3.0% w/v polysorbate 80, 0.8% w/v dextrose, and 0.54% w/v NaCl were mixed together to form the aqueous component. 20 mg of RTX was added to 100 mL of the aqueous component in a volumetric flask. The aqueous component was added to about 80% volume, and then it was sonicated to dissolve all the solids. The full mixture in the flask was diluted to volume with the aqueous component and this was mixed by an inversion process. The full formulation was filtered through a 0.2 μm PTFE filter.

A formulation according to Formulation 11 was prepared using 200 μg RTX, 20 mg Polysorbate 80 (using commercially-available Tween (C) 80); 5.4 mg of sodium chloride, 50 mg of dextrose, and a 30 mM aqueous phosphate buffer, water (WFI) to 1 mL.

### EXAMPLE 2: SOLUBILITY COMPARISON

Independently of the formulations described in Example 1, a group of 12 surfactants was tested to compare the recovery of RTX based on HPLC analysis of samples following ambient and cold (5° C.) storage. Table 2 shows the percent recovery for the different solvents tested:

TABLE 2
Solubility of RTX in Various Solutions

| | Surfactant | % Recovery | % Recovery |
|---|---|---|---|
| Solution | % (w/v) | T_{Ambient} | T_{5° C.} |
| Water | NA | 0.0 | 0.0 |
| 95% Ethanol | NA | 98.4 | 99.8 |
| n-Dodecyl-β-maltoside | 0.5 | 20.9 | 21.5 |
| Sodium 2-(diethylhexyl) | 0.5 | 3.1 | 4.4 |
| sulfosuccinate | | | |
| Sodium dodecylsulfate | 0.5 | 24.0 | 12.3 |
| Tocopheryl-polyethylene | 0.1 | 0.0 | 0.0 |
| glycol succinate | | | |
| Tween 80 | 0.01 | 0.0 | 0.0 |
| Tween 80 | 0.05 | 0.4 | 0.6 |
| Tween 80 | 0.1 | 2.7 | 3.1 |
| Tween 80 | 0.5 | 19.0 | 20.2 |
| Tween 80 | 1.0 | 12.6 | 13.4 |
| Tween 20 | 0.1 | 1.8 | 1.9 |

The study showed insolubility in water. Further, none of the aqueous surfactant solutions demonstrated recovery approaching ethanol, which reported ambient recovery of 98.4% and cold temperature recovery of 99.8%. The next closest percent recovery was just 24.0% for sodium dodecylsulfate solution, and 20.2% for 0.5% Tween 80. Example 2 demonstrates that it is difficult to achieve aqueous solubility of RTX in a non-alcoholic solvent. Many common solvents fail to provide a usable solution. Example 2 further demonstrates that RTX is not soluble in an unmodified aqueous solution.

### EXAMPLE 3: PURITY AND POTENCY OF RTX SOLUTIONS

Formulations 1-10 of Table 1 were also tested to measure the purity and potency of the RTX. These measurements provide an indication of the stability of the RTX in solution, demonstrating that the RTX remains in solution when the tested aliquots were drawn. The tests were performed at the initial time of preparation of the solution, and then subsequently at set time periods following preparation of the solutions. Formulations 1 through 10 (above) were studied in Example 3.

For purity, potency, and related substances testing, approximately 2 mL of each formation was filtered through 0.2 μm, 13 mm, PTFE filter, and approximately the first 1 mL was discarded. The unfiltered samples were also analyzed, as shown below. All samples were analyzed by HPLC with an injection volume of 50 μL. Table 3.1 shows purity and potency results with and without filtration.

TABLE 3.1
RTX Formulation Assay Testing Summary (t = 0)

| | Unfiltered | | Filtered | |
|---|---|---|---|---|
| | Purity | Potency | Purity | Potency |
| Formulation | (%) | (%) | (%) | (%) |
| 1 | 99.10 | 97.22 | 99.06 | 97.79 |
| 2 | 99.32 | 96.46 | 99.19 | 97.61 |
| 3 | 99.24 | 98.72 | 99.13 | 99.62 |
| 4 | 99.21 | 93.15 | 99.18 | 99.19 |
| 5 | 99.02 | 96.37 | 99.03 | 96.84 |
| 6 | 98.97 | 97.37 | 98.93 | 97.47 |
| 7 | 99.15 | 98.35 | 98.92 | 98.53 |
| 8 | 99.25 | 97.65 | 99.21 | 98.86 |
| 9 | 99.26 | 95.63 | 99.21 | 97.70 |
| 10 | 99.21 | 96.25 | 99.16 | 97.38 |

In a further analysis, 100 μL of each formulation was diluted 1:10 in cerebrospinal fluid (CSF) and tested for appearance, potency, purity, and related substances. All solutions remained visually clear after dilution. The samples were filtered through 0.2 μm, 13 mm, PTFE filter, discarding the first 800 μL. All samples were analyzed at an injection volume of 50 μL. The results are shown in Table 3.2:

TABLE 3.2
RTX Solution Testing in CSF

| Formulation | Purity (%) | Potency (%) |
|---|---|---|
| 1 | 99.44 | 134.48 |
| 2 | 99.32 | 93.65 |
| 3 | 99.07 | 109.51 |
| 4 | 98.98 | 62.68 |
| 5 | 98.95 | 130.19 |
| 6 | 99.20 | 131.16 |
| 7 | 99.40 | 133.71 |
| 8 | 99.66 | 96.23 |
| 9 | 99.14 | 94.37 |
| 10 | 98.82 | 77.40 |

The study demonstrated high purity and potency. In general, high potency values (e.g., values exceeding 100%) are believed to reflect a filter compatibility issue for CSF filtration sample at low concentration.

### EXAMPLE 4: RTX STABILITY OVER TIME

In a further study, samples as described above were stored and analyzed after 0.5 and 1 months in storage. Results for Potency at 0.5 and 1 month appear in Table 4.1 and 4.2.

TABLE 4.1
RTX Formulations Potency Summary t = 0.5 month

| | Potency (%) | | | | | |
|---|---|---|---|---|---|---|
| | | | | 25° C./ | 40° C./ | |
| Form. No. | t = 0 | −20° C. | 5° C. | 60% RH | 75% RH | 60° C. |
| 1 | 97.8 | 94.8 | 91.8 | 85.6 | 81.3 | 80.2 |
| 2 | 96.9 | 91.5 | 90.9 | 90.4 | 68.3 | 53.3 |
| 3 | 99.8 | 95.7 | 95.7 | 90.0 | 78.2 | 50.9 |
| 4 | 91.4 | 88.7 | 79.1 | 61.7 | 57.2 | 25.8 |
| 5 | 96.9 | 78.3 | 91.6 | 87.4 | 88.2 | 78.0 |
| 6 | 97.9 | 77.9 | 91.4 | 82.5 | 66.0 | 46.7 |
| 7 | 99.5 | 78.6 | 93.2 | 85.7 | 72.5 | 48.9 |
| 8 | 98.7 | 68.9 | 92.7 | 88.1 | 68.1 | 52.3 |
| 9 | 97.0 | 73.2 | 92.1 | 89.4 | 77.3 | 65.2 |
| 10 | 96.7 | 78.5 | 91.8 | 88.8 | 75.1 | 61.9 |

TABLE 4.2
RTX Prototype Formulations Potency Summary t = 1 month

| | Potency (%) | | | | | |
|---|---|---|---|---|---|---|
| | | | | 25° C./ | 40° C./ | |
| Form. No. | t = 0 | −20° C. | 5° C. | 60% RH | 75% RH | 60° C. |
| 1 | 97.8 | 97.1 | 95.3 | 82.9 | 85.2 | 73.2 |
| 3 | 99.8 | 100.5 | 99.4 | 89.2 | 72.0 | 33.1 |
| 5 | 96.9 | 96.3 | 94.8 | 88.3 | 90.0 | 68.0 |

The data in Table 4.1 shows that formulations with mannitol maintain pH more consistently than formulations with dextrose, as may be seen by comparison of formulation 1 to formulation 7; formulation 2 to formulation 8; formulation 5 to formulation 6; and formulation 9 to formulation 10.

Further, the results in Table 4.1 demonstrate that the best storage at −20° C. was achieved by Formulations 1 and 3. At 5° C., all formulations, except for formulation 4, gave better than 90% potency with formulation 3 giving the highest potency. For 25° C./60% RH, formulations 3 and 5 gave the best potency. For 40° C./75% RH, formulation 5 gave the best potency. For 60° C., formulations 1 and 5 gave the best potency.

Purity was also tested after 0.5 and 1 month. These results are shown in Tables 4.3 and 4.4.

TABLE 4.3
RTX Formulations Purity Summary t = 0.5 month

| | Purity (%) | | | | | |
|---|---|---|---|---|---|---|
| | | | | 25° C./ | 40° C./ | |
| Form. No. | t = 0 | −20° C. | 5° C. | 60% RH | 75% RH | 60° C. |
| 1 | 99.21 | 99.42 | 98.86 | 93.48 | 93.25 | 95.09 |
| 2 | 99.35 | 99.37 | 99.39 | 97.10 | 95.29 | 90.77 |
| 3 | 99.40 | 99.69 | 99.90 | 95.54 | 88.60 | 78.19 |
| 4 | 99.46 | 99.33 | 98.64 | 94.10 | 89.79 | 81.75 |
| 5 | 99.41 | 99.57 | 99.01 | 95.44 | 96.77 | 96.34 |
| 6 | 99.26 | 99.51 | 98.39 | 92.53 | 81.40 | 66.55 |
| 7 | 99.40 | 99.62 | 98.81 | 93.72 | 85.54 | 68.01 |
| 8 | 99.29 | 99.52 | 99.32 | 97.56 | 94.15 | 89.13 |
| 9 | 99.28 | 99.52 | 99.41 | 99.06 | 98.12 | 84.17 |
| 10 | 99.37 | 99.61 | 99.12 | 98.18 | 95.84 | 92.49 |

TABLE 4.4
RTX Prototype Formulations Purity Summary t = 1 month

| | Purity (%) | | | | | |
|---|---|---|---|---|---|---|
| | | | | 25° C./ | 40° C./ | |
| Form. No. | t = 0 | −20° C. | 5° C. | 60% RH | 75% RH | 60° C. |
| 1 | 99.21 | 99.57 | 98.02 | 89.22 | 93.23 | 93.49 |
| 3 | 99.40 | 99.66 | 98.81 | 92.41 | 84.76 | 73.92 |
| 5 | 99.41 | 99.38 | 98.36 | 94.05 | 94.70 | 94.73 |

The results in Table 4.3 demonstrate that at −20° C. all formulations showed comparable purity to t =0 data. At 5° C., formulations 2, 3, 8, and 9 shows the best purity results with the other formulations showing a 0.2-0.9% drop in purity. For 25° C./60% RH, formulations 3 and 5 showed the best response, with about 4% drop in purity. Table 4.4 shows the corresponding results measured for certain formulations after 1 month.

### EXAMPLE 5: PH STABILITY

Formulations 1-10 were also studied to determine their pH upon preparation (t=0) and after 0.5 and 1 month. These results are shown in Tables 5.1 and 5.2.

TABLE 5.1
RTX Formulation pH Summary t - 0.5 month

| | | | | 25° C./ | 40° C./ | |
|---|---|---|---|---|---|---|
| Form. No. | pH (t = 0) | −20° C. | 5° C. | 60% RH | 75% RH | 60° C. |
| 1 | 7.04 | 7.05 | 7.04 | 7.04 | 6.98 | 6.74 |
| 2 | 6.31 | 6.28 | 6.29 | 6.27 | 6.27 | 6.00 |
| 3 | 6.83 | 6.81 | 6.82 | 6.80 | 6.79 | 6.66 |
| 4 | 6.82 | 6.83 | 6.83 | 6.84 | 6.84 | 6.78 |
| 5 | 7.04 | 7.00 | 7.00 | 7.01 | 6.98 | 6.71 |
| 6 | 7.04 | 7.01 | 7.00 | 7.01 | 6.99 | 6.94 |
| 7 | 7.05 | 7.04 | 7.04 | 7.02 | 6.98 | 6.87 |
| 8 | 6.22 | 6.23 | 6.25 | 6.25 | 6.26 | 6.23 |
| 9 | 6.37 | 6.30 | 6.35 | 6.33 | 6.29 | 5.41 |
| 10 | 6.31 | 6.29 | 6.30 | 6.30 | 6.28 | 6.24 |

TABLE 5.2
RTX Formulations pH Summary t = 1 month

| Form. | | | | 25° C./ | 40° C./ | 60° C. | |
|---|---|---|---|---|---|---|---|
| # | t = 0 | −20° C. | 5° C. | 60% RH | 75% RH | 0.5 month | 1 month |
| 1 | 7.04 | 7.01 | 7.07 | 7.05 | 6.97 | 6.74 | 6.56 |
| 3 | 6.83 | 6.76 | 6.80 | 6.83 | 6.79 | 6.66 | 6.58 |
| 5 | 7.04 | 7.04 | 7.05 | 7.03 | 6.93 | 6.71 | 6.44 |

As shown by the foregoing Table 5.1 and 5.2, the formulations exhibited good stability of pH over time. Especially with regard to Table 5.2, the samples stored at less than or equal to 40°° C. showed no significant shift in pH. For formulations stored at 60° C., each formulation showed further decreases in pH compared to the t =0.5 month results.

What is claimed is:

1. A non-alcoholic formulation of RTX comprising from about 10 μg/mL to about 200 μg/mL RTX solubilized in a solubilizing agent, a monosaccharide or sugar alcohol, and a buffer solution, wherein the formulation has a pH from about 6.5 to about 7.5.

2. The non-alcoholic formulation of RTX of claim 1, wherein the solubilizing agent is selected from the group consisting of PEG, polysorbate and cyclodextrin, or combinations thereof.

3. The non-alcoholic formulation of RTX of claim 1, wherein the formulation comprises from about 25-50 μg/mL RTX.

4. The non-alcoholic formulation of RTX of claim 1, wherein the monosaccharide or sugar alcohol is selected from the group consisting of dextrose and mannitol, or combinations thereof.

5. The non-alcoholic formulation of RTX of claim 1, wherein the saline buffer is selected from the group consisting of a phosphate buffer, an acetate buffer, and a citrate buffer, or combinations thereof.

6. The non-alcoholic formulation of RTX of claim 1, further comprising an antioxidant.

7. The non-alcoholic formulation of RTX of claim 6, wherein the antioxidant is selected from the group consisting of ascorbic acid, citric acid, potassium bisulfate, sodium bisulfate acetone sodium bisulfate, monothioglycerol, potassium metabisulfite, and sodium metabisulfite, or combinations thereof.

8. The non-alcoholic formulation of RTX of claim 2, wherein the solubilizing agent is selected from the group consisting of PEG (0-40%), polysorbate (0-5%) and cyclodextrin (0-5%), or combinations thereof.

9. The non-alcoholic formulation of RTX of claim 1, comprising from about 10 μg/mL to about 200 μg/mL RTX solubilized in polysorbate 80, dextrose, and a phosphate buffer solution, wherein the formulation has a pH from about 6.5 to about 7.5.

10. The non-alcoholic formulation of RTX of claim 9, comprising 200 μg/mL RTX solubilized in 0.03% v/v polysorbate 80, 0.05% w/v dextrose, and 30 mM phosphate buffer solution, wherein the formulation has a pH of about 7.2.
